(19) United States
(12) Patent Application Publication
(10) Pub. No.: US 2023/0123017 A1
(43) Pub. Date: Apr. 20, 2023

(54) SYSTEMS AND METHODS FOR LABEL-FREE TRACKING OF HUMAN SOMATIC CELL REPROGRAMMING

(71) Applicant: WISCONSIN ALUMNI RESEARCH FOUNDATION, Madison, WI (US)

(72) Inventors: Melissa C. Skala, Middleton, WI (US); Kaivalya Molugu, Madison, WI (US); Krishanu Saha, Madison, WI (US)

(21) Appl. No.: 17/968,027

(22) Filed: Oct. 18, 2022

Related U.S. Application Data
(60) Provisional application No. 63/257,034, filed on Oct. 18, 2021.

(51) Int. Cl. C12N 15/10; G16B 20/00; G16B 40/10; G01N 21/64

(52) U.S. Cl. CPC C12N 15/1055; G16B 20/00; G16B 40/10; G01N 21/6486

(57) ABSTRACT

Systems and methods for identifying a current reprogramming status and for predicting a future reprogramming status for reprogramming intermediate cells (i.e., somatic cells undergoing reprogramming) are provided. Label-free autofluorescence measurements are combined with machine learning techniques to provide highly accurate identification of current reprogramming status and prediction of future reprogramming status. The identification of current reprogramming status utilizes metabolic endpoints from the autofluorescence data set. The prediction of future reprogramming status utilizes a pseudotime line constructed from autofluorescence data of reprogramming intermediate cells having a known reprogramming status.

### CROSS-REFERENCE TO RELATED APPLICATIONS

This application is related to, claims priority to, and incorporated herein by reference for all purposes U.S. Provisional Patent Application No. 63/257,034, filed Oct. 18, 2021.

### STATEMENT REGARDING FEDERALLY FUNDED RESEARCH

This invention was made with government support under GM119644 awarded by the National Institutes of Health. The government has certain rights in the invention.

### BACKGROUND

Derivation of patient-specific induced pluripotent stem cells (iPSCs) from their somatic cells via reprogramming generates a unique self-renewing cell source for disease modeling, drug discovery, toxicology, and personalized cell therapies. These cells carry the genome of the patient, facilitating elucidation of the genetic causes of disease, and are immunologically matched to the patient, facilitating the engraftment of any cell therapies developed from these cells.

A need exists for new platforms for biomanufacturing of iPSCs that are integration-free, fast, efficient, scalable, and easily transferrable to GMP-compliant conditions.

### SUMMARY

In one aspect, the present disclosure provides a somatic cell reprogramming tracking device. The device includes a cell analysis observation zone, an autofluorescence spectrometer, a processor, and a non-transitory computer-readable medium. The cell analysis observation zone is adapted to receive a reprogramming intermediate cell and to present the reprogramming intermediate cell for individual autofluorescence interrogation. The autofluorescence spectrometer is configured to acquire an autofluorescence data set for the reprogramming intermediate cell located in the cell analysis observation zone. The autofluorescence spectrometer includes a light source, a photon-counting detector, and photon-counting electronic. The processor is in electronic communication with the autofluorescence spectrometer. The non-transitory computer-readable medium is accessible to the processor and has stored thereon instructions. The instructions, when executed by the processor, cause the processor to: a) receive the autofluorescence data set; and b) identify a current reprogramming status of the reprogramming intermediate cell based on a current reprogramming prediction, wherein the current reprogramming prediction is computed using at least a portion of the autofluorescence data set, wherein the current reprogramming prediction is computed using at least one metabolic endpoint of the autofluorescence data set and optionally at least one nuclear parameter as an input, wherein the at least one metabolic endpoint includes flavin adenine dinucleotide (FAD) mean fluorescence lifetime (τₘ), the FAD shortest lifetime amplitude component (α₁), FAD shortest fluorescence lifetime component (τ₁), FAD longest fluorescence lifetime component (τ₂), or a combination thereof.

In another aspect, the present disclosure provides a method of characterizing somatic cell reprogramming progression. The method includes the following steps: a) optionally receiving a population of reprogramming intermediate cells having unknown reprogramming status; b) acquiring an autofluorescence data set from a reprogramming intermediate cell of the population of reprogramming intermediate cells; and c) identifying a current reprogramming status of the reprogramming intermediate cell based on a current reprogramming prediction, wherein the current reprogramming prediction is computed using at least a portion of the autofluorescence data set, wherein the current reprogramming prediction is computed using at least one metabolic endpoint of the autofluorescence data set and optionally at least one nuclear parameter as an input, wherein the at least one metabolic endpoint includes flavin adenine dinucleotide (FAD) mean fluorescence lifetime (τₘ), the FAD shortest lifetime amplitude component (α₁), FAD shortest fluorescence lifetime component (τ₁), FAD longest fluorescence lifetime component (τ₂), or a combination thereof.

In another aspect, the present disclosure provides a method of characterizing somatic cell reprogramming progression. The method includes the following steps: a) receiving a population of reprogramming intermediate cells having unknown reprogramming status; b) acquiring an autofluorescence data set for each reprogramming intermediate cell of the population of reprogramming intermediate cells, each autofluorescence data set including autofluorescence lifetime information; and either: c1) physically isolating a first portion of the population of reprogramming intermediate cells from a second portion of the population of reprogramming intermediate cells based on a current reprogramming prediction, wherein each reprogramming intermediate cell of the population of reprogramming intermediate cells is placed into the first portion when the current reprogramming prediction exceeds a predetermined threshold and into the second portion when the current reprogramming prediction is less than or equal to the predetermined threshold; or c2) generating a report including the current reprogramming prediction, the report optionally identifying a proportion of the population of reprogramming intermediate cells having the current reprogramming prediction that exceeds the predetermined threshold, wherein the current reprogramming prediction is computed using at least one metabolic endpoint of the autofluorescence data set and optionally at least one nuclear parameter as an input, wherein the at least one metabolic endpoint includes flavin adenine dinucleotide (FAD) mean fluorescence lifetime (τₘ), the FAD shortest lifetime amplitude component (α₁), FAD shortest fluorescence lifetime component (τ₁), FAD longest fluorescence lifetime component (τ₂), or a combination thereof.

In another aspect, the present disclosure provides a method of making a pseudotime reprogramming pathway map. The method includes the following steps: a) receiving autofluorescence data sets and optionally nuclear data sets for a plurality of reprogramming intermediate cells, the autofluorescence data sets corresponding to pseudotime points along a pseudotime line of reprogramming; b) constructing pseudotime single-cell trajectories for each of the plurality of reprogramming intermediate cells based on the received autofluorescence data sets associated with each of the plurality of reprogramming intermediate cells, the pseudotime single-cell trajectories each including a current reprogramming prediction associated with each of the predetermined pseudotime points, the current reprogramming prediction is computed using at least a portion of the autofluorescence data sets and optionally using at least a portion of the nuclear data sets, wherein the current reprogramming prediction is computed using at least one metabolic endpoint of at least one of the autofluorescence data sets and optionally at least one nuclear parameter of at least one of the nuclear data sets as an input, wherein the at least one metabolic endpoint includes flavin adenine dinucleotide (FAD) mean fluorescence lifetime (τₘ), the FAD shortest lifetime amplitude component (α₁), FAD shortest fluorescence lifetime component (τ₁), nicotinamide adenine dinucleotide and/or reduced nicotinamide dinucleotide phosphate adenine dinucleotide (NAD(P)H) shortest lifetime amplitude component (α₁), NAD(P)H shortest fluorescence lifetime component (τ₁), NAD(P)H longest fluorescence lifetime component (τ₂), or a combination thereof; c) compiling the constructed pseudotime single-cell trajectories into a single compiled data set; d) identifying clusters, branching events, and/or disconnected branches within the single compiled data set; and e) identifying correlation within the single compiled data set between the clusters, branching events, and/or disconnected branches and current or future reprogramming status, thereby producing the pseudotime reprogramming pathway map for use in predicting future reprogramming based on the correlation.

### BRIEF DESCRIPTIONS OF THE DRAWINGS AND APPENDIX

FIG. 1 is a flowchart illustrating a method, in accordance with an aspect of the present disclosure.

FIG. 2 is a flowchart illustrating a method, in accordance with an aspect of the present disclosure.

FIG. 3 is a flowchart illustrating a method, in accordance with an aspect of the present disclosure.

FIG. 4 is a flowchart illustrating a method, in accordance with an aspect of the present disclosure.

FIG. 5 is a block diagram of a device, in accordance with an aspect of the present disclosure.

FIG. 6 is a plot of trajectory of reprogramming EPCs constructed from the metabolic and nuclear parameters based on UMAP dimension reduction using Monocle showing four branch points colored by cell type.

FIG. 7 is a plot of trajectory of reprogramming EPCs constructed from the metabolic and nuclear parameters based on UMAP dimension reduction using Monocle showing four branch points colored by pseudotime.

FIG. 8 is a set of Monocle UMAP plots showing clustering of reprogramming EPCs.

### DETAILED DESCRIPTION

Before the present invention is described in further detail, it is to be understood that the invention is not limited to the particular embodiments described. It is also understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

Specific structures, devices and methods relating to modifying biological molecules are disclosed. It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

As used herein, the term “FAD” refers to flavin adenine dinucleotide.

As used herein, the term “memory” includes a non-volatile medium, e.g., a magnetic media or hard disk, optical storage, or flash memory; a volatile medium, such as system memory, e.g., random access memory (RAM) such as DRAM, SRAM, EDO RAM, RAMBUS RAM, DR DRAM, etc.; or an installation medium, such as software media, e.g., a CD-ROM, or floppy disks, on which programs may be stored and/or data communications may be buffered. The term “memory” may also include other types of memory or combinations thereof.

As used herein, the term “NAD(P)H” refers to reduced nicotinamide adenine dinucleotide and/or reduced nicotinamide dinucleotide phosphate.

As used herein, “nuclear parameter” refers to a measured geometric or clustering property of a nucleus of a cell of interest as determined by analyzing an acquired image of the cell of interest.

As used herein, the term “processor” may include one or more processors and memories and/or one or more programmable hardware elements. As used herein, the term “processor” is intended to include any of types of processors, CPUs, GPUs, microcontrollers, digital signal processors, or other devices capable of executing software instructions.

As used herein, the terms “pseudotime”/“pseudotime line” refers to a progression dimension along the cell reprogramming pathway. Pseudotime is related to time in that the progression is directional in the same fashion as time, but is different than time in that it is not related to fixed passage of time. Two different cells can take different lengths of actual time to traverse the same length of pseudotime.

As used herein, the term “redox ratio” or “optical redox ratio” refers to a ratio of NAD(P)H fluorescence intensity to FAD fluorescence intensity; a ratio of FAD fluorescence intensity to NAD(P)H fluorescence intensity; a ratio of NAD(P)H fluorescence intensity to any arithmetic combination including FAD fluorescence intensity; or a ratio of FAD fluorescence intensity to any arithmetic combination including NAD(P)H fluorescence intensity. In certain cases, the redox ratio or optical redox ratio refers to a ratio of NAD(P)H fluorescence intensity to the sum of NAD(P)H and FAD fluorescence intensity.

As used herein, “somatic cells” refers to any non-reproductive cell. In this disclosure, the term somatic cell can refer to a single cell that is subjected to reprogramming. A somatic cell that is undergoing reprogramming can be identified by a single name, a “reprogramming intermediate (IM) cell”, with the understanding that the cell type of that cell may be changing.

Autofluorescence endpoints include photon counts/intensity and fluorescence lifetimes. The fluorescence lifetime of cells can be a single value, the mean fluorescence lifetime, or compromised from the lifetime values of multiple subspecies with different lifetimes. In this case, multiple lifetimes and lifetime component amplitude values are extracted. Both NAD(P)H and FAD can exist in quenched (short lifetime) and unquenched (long lifetime) configurations; therefore, the fluorescence decays of NAD(P)H and FAD are fit to two components. Generally, NADH and FAD fluorescence lifetime decays are fit to a two component exponential decay, I(t)=α₁e^{-t/τ1}+α₂e^{-t/τ2}+C, where I(t) is the fluorescence intensity as a function of time, t, after the laser pulse, α₁ and α₂ are the fractional contributions of the short and long lifetime components, respectively (i.e., α₁+α₂=1), τ₁ and τ₂ are the short and long lifetime components, respectively, and C accounts for background light. However, the lifetime decay can be fit to more components (in theory any number of components, although practically up to ˜5-6) which would allow quantification of additional lifetimes and component amplitudes. By convention lifetimes and amplitudes are numbered from short to long, but this could be reversed. A mean lifetime can be computed from the lifetime components, (τₘ=α₁τ₁+α₂τ₂ . . . ). Fluorescence lifetimes and lifetime component amplitudes can also be approximated from frequency domain data and gated cameras/detectors. For gated detection, α₁ could be approximated by dividing the detected intensity at early time bins by later time bins. Alternatively, fluorescence anisotropy can be measured by polarization-sensitive detection of the autofluorescence, thus identifying free NAD(P)H as the short rotational diffusion time in the range of 100-700 ps.

FADτ₁ refers to the contribution of bound FAD and is the shortest lifetime that is not dominated (i.e., greater than 50%) by instrument response and/or scattering. FADτ₁ is the contribution associated with FAD lifetime values from 50-1500 ps, from 50-1000 ps, or from 50-600 ps. For clarity, a claim herein including features related to a “shortest” lifetime cannot be avoided by defining the lifetime values to include a sacrificial shortest lifetime that is dominated by instrument response and/or scattering.

FADτ₁ refers to the bound FAD lifetime and is the shortest lifetime that is not dominated (i.e., greater than 50%) by instrument response and/or scattering. FADτ₂ is the FAD lifetime values from 50-1500 ps, from 50-1000 ps, or from 50-600 ps. For clarity, a claim herein including features related to a “shortest” lifetime cannot be avoided by defining the lifetime values to include a sacrificial shortest lifetime that is dominated by instrument response and/or scattering.

FADτ₂ refers to the free FAD lifetime and is the longest lifetime that is not dominated (i.e., greater than 50%) by instrument response and/or scattering. FADτ₂ is the FAD lifetime values from 1000-4000 ps, from 1000-3000 ps, or from 1500-3000 ps. For clarity, a claim herein including features related to a “longest” lifetime cannot be avoided by defining the lifetime values to include a sacrificial shortest lifetime that is dominated by instrument response and/or scattering.

FADτₘ=α₁·τ₁+(1−α₁)·τ₂

The various aspects may be described herein in terms of various functional components and processing steps. It should be appreciated that such components and steps may be realized by any number of hardware components configured to perform the specified functions.

### Methods

This disclosure provides a variety of methods. It should be appreciated that various methods are suitable for use with other methods. Similarly, it should be appreciated that various methods are suitable for use with the systems described elsewhere herein. When a feature of the present disclosure is described with respect to a given method, that feature is also expressly contemplated as being useful for the other methods and systems described herein, unless the context clearly dictates otherwise.

The methods described herein include two different types of predictions regarding the reprogramming status of a given reprogramming intermediate cell. First, there is a current reprogramming prediction, which provides a computer-generated prediction for the current state of reprogramming in a given reprogramming intermediate cell of interest. For example, the current reprogramming prediction may indicate that a given reprogramming intermediate cell has been reprogrammed to an induced pluripotent stem cell. Second, there is a future reprogramming prediction, which provides a computer-generated prediction for the future state of reprogramming in a given reprogramming intermediate cell of interest based on a pseudotime reprogramming pathway map (discussed in greater detail below). Optionally, the prediction for the future state can also be based on the current reprogramming prediction. In other words, the future reprogramming prediction may indicate that a given reprogramming intermediate cell is likely or unlikely to eventually reprogram into an iPSC. This future prediction is based on a machine learning analysis of a large population of cells undergoing reprogramming, which identified that cells that fall into a given cluster on the pseudotime reprogramming pathway map have a given probability of taking the various possible reprogramming pathways that are available.

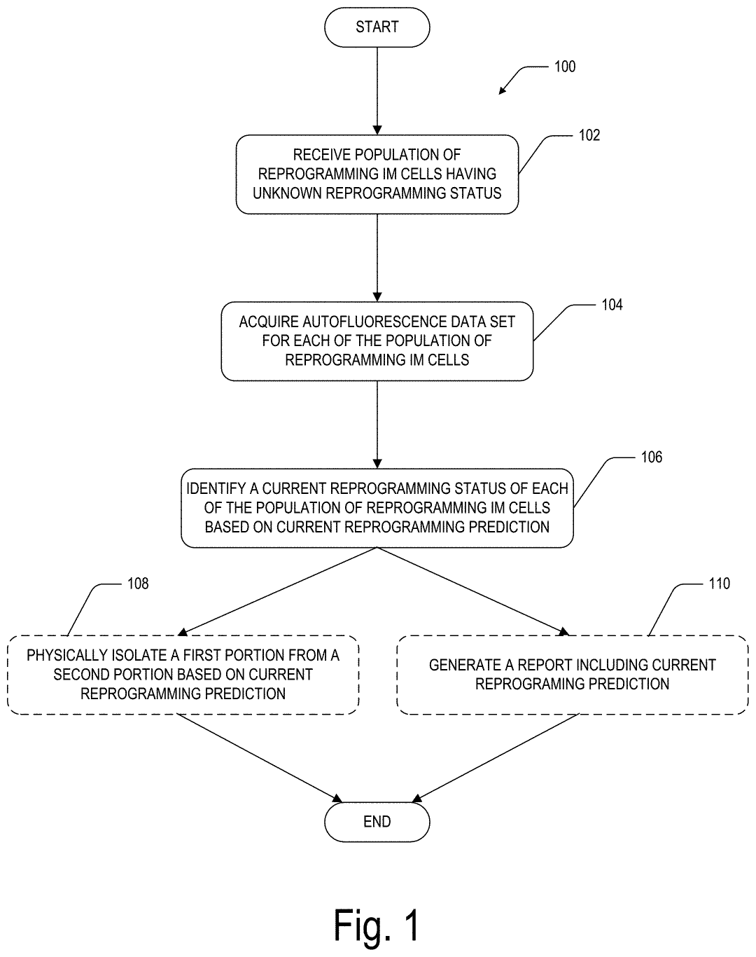

Referring to FIG. 1, the present disclosure provides a method 100 of characterizing somatic cell reprogramming progression. At process block 102, the method 100 optionally includes receiving a population of reprogramming intermediate cells having unknown reprogramming status. The population of reprogramming intermediate cells can itself be contained within a broader population of cells that includes some cells that are not reprogramming intermediate cells. At process block 104, the method 100 includes acquiring an autofluorescence data set for each reprogramming intermediate cell of the population of reprogramming intermediate cells. At process block 106, the method 100 includes identifying a current reprogramming status of each of the reprogramming intermediate cells based on a current reprogramming prediction. The current reprogramming prediction is computed using at least a portion of the autofluorescence data set. The current reprogramming prediction is computed using at least one metabolic endpoint of the autofluorescence data set and optionally using at least one nuclear parameter as an input. The at least one metabolic endpoint includes FADτₘ, FADτ₁, FADτ₂, FADα₁ or a combination thereof. Following process block 106, the method 100 can proceed to process block 108 or 110, depending on the desired outcome. In some cases, the method 100 proceeds to process block 108 and process block 110, in either order. While process blocks 108 and 110 are both illustrated and described as optional, the method 100 includes either process block 108 or process block 110. At optional process block 108, the method 100 optionally includes physically isolating a first portion of the population of reprogramming intermediate cells from a second portion of the population of reprogramming intermediate cells based on a current reprogramming prediction, wherein each reprogramming intermediate cell of the population of reprogramming intermediate cells is placed into the first portion when the current reprogramming prediction exceeds a predetermined threshold and into the second portion when the current reprogramming prediction is less than or equal to the predetermined threshold. At optional process block 110, the method 100 optionally includes generating a report including the current reprogramming prediction. The report optionally includes identifying a proportion of the population of reprogramming intermediate cells having a current reprogramming prediction that exceeds a predetermined threshold.

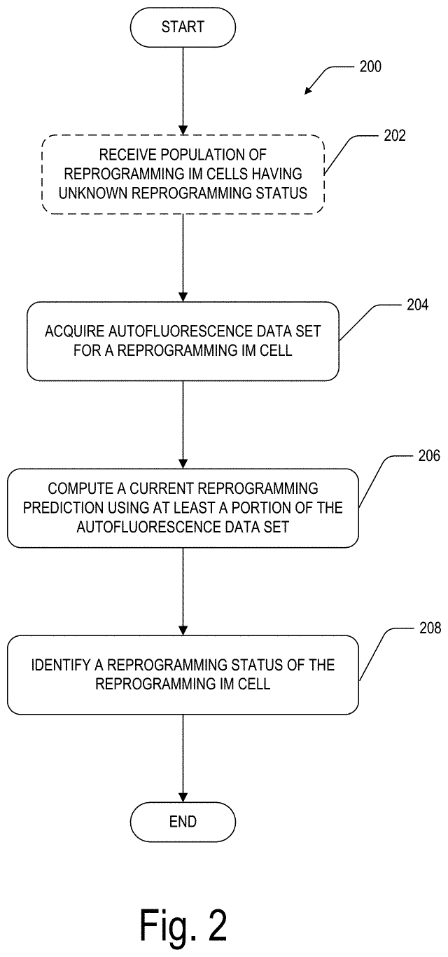

Referring to FIG. 2, the present disclosure provides a method 200 of characterizing reprogramming intermediate cell reprogramming status. At optional process block 202, the method 200 optionally includes receiving a population of reprogramming intermediate cells having unknown reprogramming status. At process block 204, the method 200 includes acquiring a autofluorescence data set from a reprogramming intermediate cell of the population of reprogramming intermediate cells. At process block 206, the method 200 includes computing a current reprogramming prediction using at least a portion of the autofluorescence data set. The current reprogramming prediction is computed using at least one metabolic endpoint and optionally using at least one nuclear parameter. The at least one metabolic endpoint includes FADτₘ, FADα₁, FADτ₁, FADτ₂, or a combination thereof. At process block 208, the method 200 includes identifying a current reprogramming status of the reprogramming intermediate cell based on the current reprogramming prediction.

Method 100 and method 200 are related to one another and can be utilized together. For example, method 200 can be utilized within method 100. Aspects described with respect to method 100 can be utilized in method 200, unless the context clearly dictates otherwise, and vice versa.

Method 100 and method 200 can further include identifying a future reprogramming status of one or more reprogramming intermediate cells either alone or within the population of reprogramming intermediate cells. The identifying of the future reprogramming status is based on the current reprogramming prediction and a pseudotime reprogramming pathway map that is based on machine learning analysis of acquired autofluorescence data sets for reprogramming intermediate cells having a known reprogramming status over the course of a pseudotime trajectory. In some cases, the pseudotime reprogramming pathway map is produced by method 400, as discussed below.

The autofluorescence data set acquired at process block 104 or 204 can be acquired in a variety of ways, as would be understood by one having ordinary skill in the spectroscopic arts with knowledge of this disclosure and their own knowledge from the field. For example, the autofluorescence data can be acquired from fluorescence decay data. As another example, the autofluorescence data can be acquired by gating a detector (a camera, for instance) to acquire data at specific times throughout a decay in order to approximate the autofluorescence endpoints described herein. As yet another example, a frequency domain approach can be used to measure lifetime. Alternatively, fluorescence anisotropy can be measured by polarization-sensitive detection of the autofluorescence, thus identifying free NAD(P)H as the short rotational diffusion time in the range of 100-700 ps. The specific way in which autofluorescence data is acquired is not intended to be limiting to the scope of the present invention, so long as the lifetime information necessary to determine the autofluorescence endpoints necessary for the methods described herein can be suitably measured, estimated, or determined in any fashion. One example of a suitable autofluorescence data set acquisition is described below in the Examples section.

The physical isolation operation of optional process block 108 is in response to a current reprogramming prediction determined from the acquired autofluorescence data set. If the current reprogramming prediction exceeds a predetermined threshold for a given reprogramming intermediate cell, then that reprogramming intermediate cell is placed into the first portion. If the current reprogramming prediction is less than or equal to the predetermined threshold for the given reprogramming intermediate cell, then that reprogramming intermediate cell is placed into the second portion. The result of this physical isolation is that the first portion of the population of reprogramming intermediate cells is significantly enriched in reprogramming intermediate cells having a given reprogramming status (e.g., successfully reprogrammed as iPSCs), whereas the second portion of the population of reprogramming intermediate cells is significantly depleted of reprogramming intermediate cells having that given reprogramming status.

In some cases, the physical isolation operation of optional process block 108 can include isolating cells into three, four, five, six, or more portions. In these cases, the different portions will be separated by a number of predetermined thresholds that is one less than the number of portions (i.e., three portions=two predetermined thresholds). The portion whose current reprogramming prediction exceeds all of the predetermined thresholds (i.e., exceeds the highest threshold) contains the greatest concentration of reprogramming intermediate cells with a given reprogramming status. The portion whose current reprogramming prediction fails to exceed any of the predetermined thresholds (i.e., fails to exceed the lowest threshold) contains the lowest concentration of reprogramming intermediate cells with the given reprogramming status. Using multiple predetermined thresholds can afford the preparation of portions of the population of reprogramming intermediate cells that have extremely high or extremely low concentrations of reprogramming intermediate cells with the given reprogramming status. In some cases, the physical isolation operation of optional process block 108 (or a totally separate aspect of method 100, as would be appreciated by those having ordinary skill in the cell isolation arts) can include isolating other kinds of cells, such as red blood cells or the like, or various kinds of debris so they are not included in the portions including reprogramming intermediate cells.

The current reprogramming prediction is computed using at least one metabolic endpoint of the autofluorescence data set and optionally include at least one nuclear parameters for each reprogramming intermediate cell of the population of reprogramming intermediate cells as an input. The current reprogramming prediction is computed using an equation that is generated by a machine learning process on data for a population of reprogramming intermediate cells having a known reprogramming status using the at least one metabolic endpoint and optionally the at least one nuclear parameter as a variable. In some cases, the reprogramming precition can have different predictability for different states of reprogramming (i.e., can be more predictive of iPSC state other states).

The at least one metabolic endpoint includes the FAD mean fluorescence lifetime (τₘ), the FAD shortest lifetime amplitude component (α₁), the FAD shortest fluorescence lifetime component (τ₁), the FAD longest fluorescence lifetime component (τ₂), or a combination thereof. The at least one metabolic endpoint can also optionally include one or more of the following: NAD(P)H fluorescence intensity; FAD fluorescence intensity; an optical redox ratio (i.e., NAD(P)H/[NAD(P)H+FAD], see definition above); NAD(P)H shortest lifetime amplitude component or NAD(P)H α₁; NAD(P)H mean fluorescence lifetime or NAD(P)Hτₘ; NAD(P)H shortest fluorescence lifetime or NAD(P)Hτ₁; NAD(P)H second shortest fluorescence lifetime or NAD(P)Hτ₂.

The at least one nuclear parameter can include an area of the nucleus of the reprogramming intermediate cell, a perimeter of the nucleus of the reprogramming intermediate cell, a nuclear shape index, a mean distance of any pixel within the nucleus of the reprogramming intermediate cell to a closest pixel outside of the nucleus, a proportion of pixels located in a convex hull (i.e., the smallest convex polygon that fits around the nucleus) that are also located within the nucleus of the reprogramming intermediate cell, a proportion of pixels in a bounding box of the nucleus (i.e., the smallest rectangle that surrounds the nucleus) that are also located within the nucleus of the reprogramming intermediate cell, a total number of nuclei neighboring the reprogramming intermediate cell nucleus, a distance from the reprogramming intermediate cell nucleus to a closest neighboring nucleus, or a combination thereof.

In some cases, two, three, four, five, six, seven, eight, nine, ten, eleven, twelve, thirteen, fourteen, fifteen, sixteen, seventeen, eighteen, or more inputs are used.

The method 100 or method 200 can sort reprogramming intermediate cells into the categories of EPCs, IMs, and iPSCs based on the current reprogramming status.

The method 100 or method 200 can provide surprising accuracy of classifying somatic cell current reprogramming state. The accuracy can be at least 85%, at least 87.5%, at least 90%, at least 92.5%, at least 95%, at least 96%, at least 97%, or at least 98%. One non-limiting example of measuring the accuracy includes executing the method 100 or method 200 on a given cell with unknown current reprogramming status and then using one of the traditional methods for determining reprogramming status (which will typically be a destructive method) for a number of cells that is statistically significant.

The method 100 or method 200 can be performed without the use of a fluorescent label for binding the reprogramming intermediate cell. The method 100 or method 200 can be performed without immobilizing the reprogramming intermediate cell.

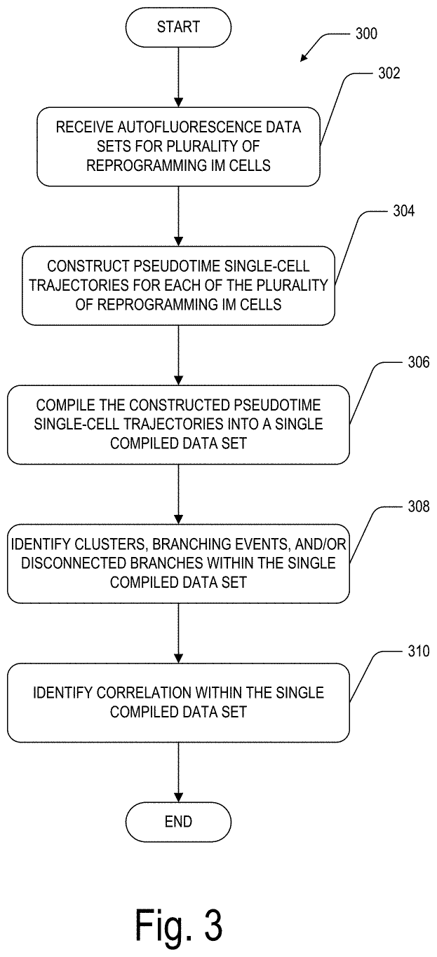

Referring to FIG. 3, the present disclosure provides a method 300 of making a pseudotime reprogramming pathway map. At process block 302, the method 300 includes receiving autofluorescence data sets for a plurality of reprogramming intermediate cells undergoing reprogramming, the autofluorescence data sets corresponding to pseudotime points along a pseudotime line of reprogramming. At process block 304, the method 300 includes constructing pseudotime single-cell trajectories for each of the plurality of reprogramming intermediate cells based on the received autofluorescence data sets associated with each of the plurality of reprogramming intermediate cells. The pseudotime single-cell trajectories each include a current reprogramming prediction associated with each of the predetermined pseudotime points. The current reprogramming prediction is computed as described herein. The current reprogramming prediction used at this step of the method 300 is computed using at least one metabolic endpoint and optionally at least one nuclear parameter as an input. The metabolic endpoint used here can include FADτₘ, FADα₁, FADτ₁, NAD(P)H α₁, NAD(P)Hτ₁, NAD(P)Hτ₂, or a combination thereof. At process block 306, the method 300 includes compiling the constructed pseudotime single-cell trajectories into a single compiled data set. At process block 308, the method 300 includes identifying clusters, branching events, and/or disconnected branches within the single compiled data set. At process block 310, the method 300 includes identifying correlation within the single compiled data set between the clusters, branching events, and/or disconnected branches and current or future reprogramming status, thereby producing the pseudotime reprogramming pathway map for use in predicting future reprogramming based on the correlation.

In this process, differentiated cells called erythroid progenitor cells or EPCs are isolated from human blood and are reprogrammed to induced pluripotent stem cells or iPSCs. Cells undergoing reprogramming are imaged for various cell metabolism and nuclear parameters using autofluorescence microscopy. These parameters are then used to train a supervised learning algorithm. This algorithm can then identify the current state of reprogramming cells i.e., starting EPCs, partially reprogrammed intermediate cells (IMs) or completely reprogrammed iPSCs, solely based on metabolic and nuclear parameters. These parameters can additionally be used to predict the future reprogramming state of cells, and this is achieved through the construction of single cell reprogramming trajectories using machine learning. Briefly, at the start of reprogramming, the differentiated cells from the blood (termed erythroid progenitor cells or EPCs) had variable metabolic and nuclear characteristics. These cells could be separated on these characteristics into three groups, termed clusters 1, 2, and 3. Single cells in cluster 2 initiate and progress through epigenetic reprogramming. However, cells in clusters 1 and 3 behave differently and do not progress through reprogramming, even though they have been exposed to the reprogramming factors.

For the cells from cluster 2 that complete reprogramming, these pluripotent stem cells, termed iPSCs, have two different types of metabolic and nuclear characteristics. These two types of iPSCs can be grouped into to two clusters: clusters 7 and 10. The cells in culture that are not fully reprogrammed into iPSCs but are undergoing reprogramming can also be distinguished on their metabolic and nuclear characteristics. These cells are called intermediate cells, IMs, and metabolic and nuclear characteristics. Among these various cell types, trajectories can be generated to infer the sequence of changes in these characteristics as cells reprogram. By following the various trajectories, we find that cells in clusters 6 and 8 notably undergo changes in metabolic and nuclear characteristics that do not end up in successful reprogramming to iPSCs. By following several other trajectories, we identify different common branches that summarize the changes that single cells undergo in culture. Points where these branches of the trajectories separate are termed branch points. There were three distinct branch points that could be identified in reprogramming cultures. Single cells that advance right at branch points 1, 2, and 3 completely reprogram to iPSCs within 25 days of reprogramming, while cells that proceed left at branch points 1 and 3 remain at the intermediate stage. Thus, the metabolic and nuclear characteristics of single cells and their changes during culture can be used to identify cells that successfully reprogram from those that do not.

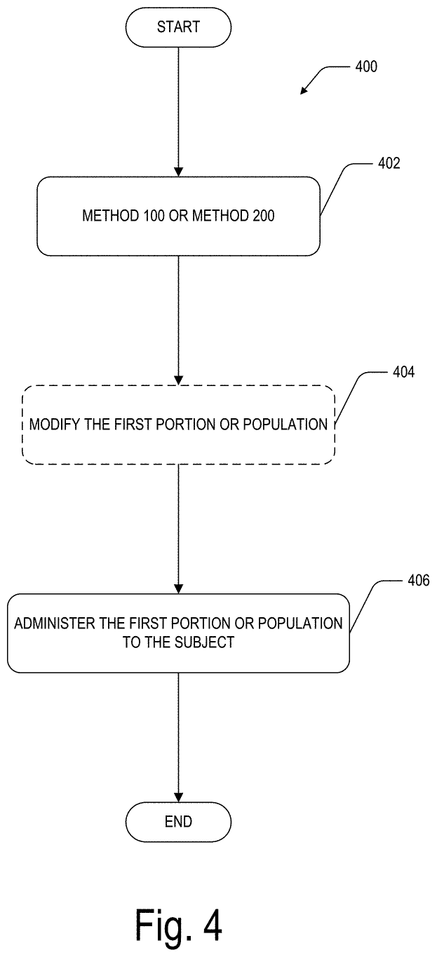

Referring to FIG. 4, the present disclosure provides a method 400 of administering reprogrammed reprogramming intermediate cells to a subject in need thereof. At process block 402, the method 400 includes the method 100 or method 200 described above, which results in a first portion of the population of reprogramming intermediate cells enriched for current or future reprogramming state (when optional process block 108 is utilized) or results in a report identifying the proportion of reprogramming intermediate cells that have a given current or future reprogramming state (when optional process block 110 is utilized). At optional process block 404, the method 400 optionally includes modifying the first portion of the population of reprogramming intermediate cells or the population of reprogramming intermediate cells. The modifying can include gene editing. At process block 406, the method 400 includes administering the first portion of the population of reprogramming intermediate cells, if the cells have been sorted, or the population of reprogramming intermediate cells, if the cells have not been sorted, to the subject.

The somatic cells from which the reprogramming intermediate cells originate can be blood cells, including peripheral blood cells, cord blood cells, bone marrow cells, and the like, skin biopsy cells, such as keratinocytes, epithelial cells, such as those shed in urine, and other cells understood to be useful in reprogramming.

The reprogramming intermediate cells can be harvested from the subject to which they are administered prior to sorting. The reprogramming intermediate cells can be either directly introduced to the subject or can undergo additional processing prior to introduction to the subject. In one case, the reprogramming intermediate cells can be modified to contain chimeric antigen receptors (CARs).

The somatic cells from which the reprogramming intermediate cells originate can be harvested from a donor.

The somatic cells can be reprogrammed according to methods understood to those having ordinary skill in the reprogramming and/or iPSC arts. Non-limiting examples of methods of reprogramming include virus factors, CRISPR, and the like. The modes of delivery for reprogramming are also intended to be non-limiting to the present disclosure. The mode of delivery can be via episomes, lentiviral, mRNA delivery, or other modes understood by those having ordinary skill in the art.

The methods described herein provided surprising results to the inventors. First, it was unclear if the acquired fluorescence data would be capable at all of classifying somatic cell reprogramming. Second, it was not clear that classifying current reprogramming status could provide insight into future reprogramming status by way of the pseudotime reprogramming pathway map described herein. Third, it was surprising that the FAD lifetime was capable of classifying somatic cell reprogramming as these parameters had not provided discriminatory ability in other contexts.

### Systems

This disclosure also provides systems. The systems can be suitable for use with the methods described herein. When a feature of the present disclosure is described with respect to a given system, that feature is also expressly contemplated as being combinable with the other systems and methods described herein, unless the context clearly dictates otherwise.

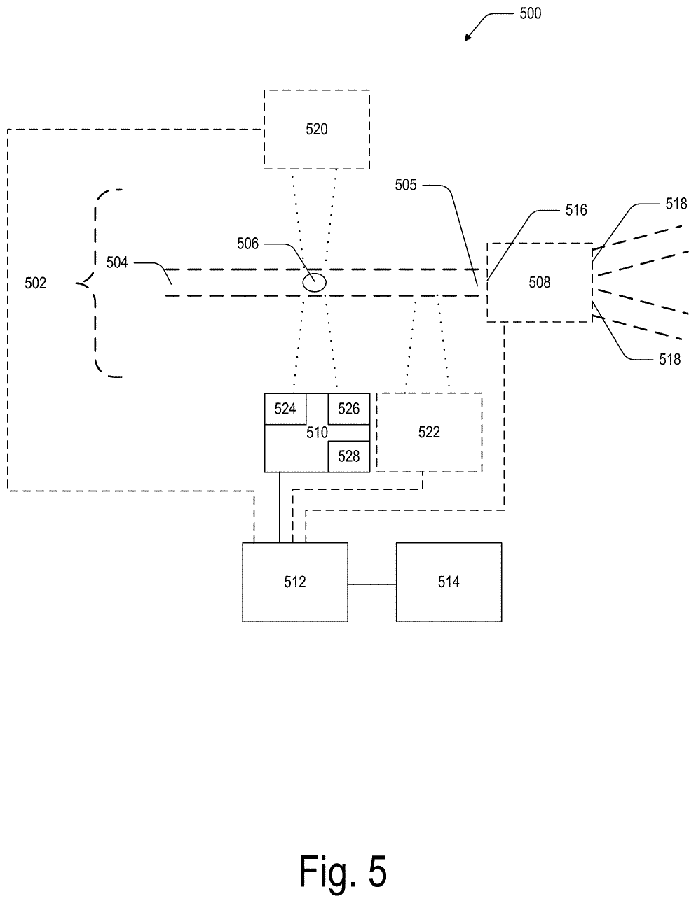

Referring to FIG. 5, the present disclosure provides a somatic cell classification device 500. The device 500 includes an observation zone 506. The observation zone 506 is adapted to receive a cell analysis pathway 502, a cell culture (not illustrated), or other device or system capable of presenting reprogramming intermediate cells for optical interrogation. The device 500 includes a processor 512 and a non-transitory computer-readable medium 514, such as a memory. In some configurations, the processor 512 can be or otherwise include a field-programmable gate array (FPGA). In configurations where the processor 512 is an FPGA, an additional processor (not shown) may be included to capture images.

The device 500 optionally includes a cell analysis pathway 502. The cell analysis pathway 502 includes an inlet 504, the observation zone 506, and an outlet 505. The device 500 optionally includes a cell sorter 508. The observation zone 506 is coupled to the inlet 504 downstream of the inlet 504 and is coupled to the outlet 505 upstream of the outlet 505. The device 500 also includes a single-cell autofluorescence spectrometer 510. The device 500 can further include an optional cell picker (not illustrated).

The inlet 504 can be any nanofluidic, microfluidic, or other cell sorting inlet. A person having ordinary skill in the art of fluidics has knowledge of suitable inlets 504 and the present disclosure is not intended to be bound by one specific implementation of an inlet 504.

The outlet can be any nanofluidic, microfluidic, or other cell sorting outlet. A person having ordinary skill in the art of fluidics has knowledge of suitable outlets 505 and the present disclosure is not intended to be bound by one specific implementation of an outlet 505.

The observation zone 506 is configured to present reprogramming intermediate cells for individual autofluorescence decay interrogation. A person having ordinary skill in the art has knowledge of suitable observation zones 506 and the present disclosure is not intended to be bound by one specific implementation of an observation zone 506.

The optional cell sorter 508 has a sorter inlet 516 and at least two sorter outlets 518. The cell sorter is coupled to the observation zone 506 via the sorter inlet 516 downstream of the observation zone 506. The cell sorter 508 is configured to selectively direct a cell from the sorter inlet 516 to one of the at least two sorter outlets 518 based on a sort signal.

The inlet 504, observation zone 506, outlet 505, and optional cell sorter 508 can be components known to those having ordinary skill in the art to be useful in high-throughput cell screening devices or flow sorters, including commercial flow sorters. The cell analysis pathway 502 can further optionally include a flow regulator, as would be understood by those having ordinary skill in the art. The flow regulator can be configured to provide flow of cells through the observation zone at a rate that allows the autofluorescence spectrometer 510 to acquire the autofluorescence data set. A useful review of the sorts of fluidics that can be used in combination with the present disclosure is Shields et al., “Microfluidic cell sorting: a review of the advances in the separation of cells from debulking to rare cell isolation,” Lab Chip, 2015 Mar. 7; 15(5): 1230-49, which is incorporated herein by reference in its entirety.

The optional cell picker can serve a similar function as the optional cell sorter 508, namely, isolating cells based on a sort signal. The cell picker can be automated. One example of a suitable cell picker includes an ALS CellCelector™, available commercially from ALS Automated Lab Solutions GmbH, Jena, Germany.

The autofluorescence spectrometer 510 includes a light source 524, a photon-counting detector 526, and photon-counting electronics 528.

The autofluorescence spectrometer 510 can be any spectrometer suitable for acquiring autofluorescence data sets as understood by those having ordinary skill in the optical arts.

Suitable light sources 524 include, but are not limited to, lasers, LEDs, lamps, filtered light, fiber lasers, and the like. The light source 524 can be pulsed, which includes sources that are naturally pulsed and continuous sources that are chopped or otherwise optically modulated with an external component.

The light source 524 can provide pulses of light having a full-width at half maximum (FWHM) pulse width that is of a duration that is adequate to achieve the spectroscopic goals described herein, as would be appreciated by one having ordinary skill in the spectroscopic arts. In some cases, the FWHM pulse width is at least 1 fs, at least 5 fs, at least 10 fs, at least 25 fs, at least 50 fs, at least 100 fs, at least 200 fs, at least 350 fs, at least 500 fs, at least 750 fs, at least 1 ps, at least 3 ps, at least 5 ps, at least 10 ps, at least 20 ps, at least 50 ps, or at least 100 ps. In some cases, the FWHM pulse width is at most 10 ns, at most 1 ns, at most 900 ps, at most 750 ps, at most 600 ps, at most 500 ps, at most 400 ps, at most 250 ps, at most 175 ps, at most 100 ps, at most 75 ps, at most 60 ps, at most 50 ps, at most 35 ps, at most 25 ps, at most 20 ps, at most 15 ps, at most 10 ps, or at most 1 ps.

The light source 524 can emit wavelengths that are tuned to the absorption of NAD(P)H and/or FAD. In some cases, the wavelength is at least 340 nm, at least 345 nm, at least 350 nm, at least 355 nm, at least 360 nm, at least 365 nm, or at least 370 nm. In some cases, the wavelength is at most 415 nm, at most 410 nm, at most 405 nm, at most 400 nm, at most 395 nm, at most 390 nm, at most 385 nm, or at most 380 nm. In some cases, the wavelength is between 360 nm and 415 nm, between 350 nm and 410 nm, or between 370 nm and 380 nm. In some cases, the wavelength is 375 nm. In some cases, the wavelength is 2 times or 3 times these wavelength values (i.e., the frequency is ½ or ⅓). It should be appreciated that pulsed light sources inherently have some degree of bandwidth, so they are never exactly monochromatic. Thus, references herein to “wavelength” refer to either a wavelength at the peak intensity or a weighted average wavelength. In some cases, the pulsed light source 524 is a UV pulsed diode laser. In some cases, the pulsed light source has a wavelength that is double the peak absorption wavelength of NAD(P)H and/or FAD, with an ultrashort pulse duration, such that fluorescence excitation is achieved through two-photon excitation events, as understood by those having ordinary skill in the optical arts.

The photon-counting detector 526 can be any detector suitably capable of detecting single photons and delivering an analog or digital output representative of the detected photons. Examples of photon-counting detectors 526 include, but are not limited to, a photomultiplier tube, a photodiode, an avalanche photodiode, a single-photon avalanche diode (SPAD), a charge-coupled device, combinations thereof, and the like.

The photon-counting electronic 528 can include electronics understood by those having ordinary skill in the art to be suitable for use with single-photon detectors 526 to produce the data sets described herein. Examples of suitable photon-counting electronics 528 include, but are not limited to, a field-programmable gate array (FPGA), a dedicated digital signal processor (DSP) with a digitizer and a time-to-digital converter, a time-correlated single photon counting (TCSPC) electronic board with time-to-amplitude and analog-to-digital converter electronics (as implemented by Becker & Hickl, Berlin, Germany), combinations thereof, and the like.

The autofluorescence spectrometer 510 can be directly (i.e., the processor 512 communicates directly with the spectrometer 510 and receives the signals) or indirectly (i.e., the processor 512 communicates with a sub-controller that is specific to the spectrometer 510 and the signals from the spectrometer 510 can be modified or unmodified before sending to the processor 512) controlled by the processor 512. Autofluorescence data sets can be acquired by known spectroscopic methods. Fluorescence lifetime images can also be acquired by known imaging methods and those acquired images can be used by the systems and methods described herein, as would be understood by those having ordinary skill in the spectroscopic arts. The device 500 can include various optical filters tuned to isolate autofluorescence signals of interest. The optical filters can be tuned to the autofluorescence wavelengths of NAD(P)H and/or FAD.

The autofluorescence spectrometer 510 can be configured to acquire the autofluorescence dataset from the detector's 526 electrical output at a repetition rate understood by those having ordinary skill in the spectroscopic arts to be suitable for providing adequate sampling to observe the dynamics disclosed herein. In some cases, the repetition rate can be at least 1 kHz, at least 5 kHz, at least 10 kHz, at least 30 kHz, at least 50 kHz, at least 100 kHz, at least 500 kHz, at least 750 kHz, at least 1 MHz, at least 4 MHz, at least 7 MHz, at least 10 MHz, at least 15 MHz, at least 20 MHz, at least 50 MHz, at least 100 MHz, at least 500 MHz, or at least 1 GHz. In some cases, the repetition rate can be at most 1 THz, at most 800 GHz, at most 500 GHz, at most 250 GHz, at most 150 GHz, at most 100 GHz, at most 70 GHz, at most 50 GHz, at most 25 GHz, at most 15 GHz, at most 10 GHz, at most 6 GHz, at most 2 GHz, at most 1 GHz, at most 750 MHz, at most 500 MHz, at most 400 MHz, at most 250 MHz, at most 175 MHz, or at most 100 MHz. While there can be downside associated with oversampling, in principle the present disclosure can function with as high of a sampling rate as can be achieved with existing technology. The repetition rates identified herein are based on the state of the art at the time the present disclosure was prepared and filed and are not intended to be limiting in the event that future developments facilitate a greater repetition rate.

The pulsed light source 524 can be configured to operate at pulse repetition rates that are adapted to acquire the needed fluorescence lifetime information. The maximum pulse repetition rate is limited by the fluorescence lifetime of the fluorophore of interest. The fluorescence decay must have fully died down by the time the next pulse of light is introduced to the sample in order to avoid ambiguity about the sources of data sets (i.e., was this particular fluorescent photon initiated by the most recent excitation pulse of light or the one preceding it?). The pulsed light source 524 can have a pulse repetition rate of up to 100 MHz, up to 80 MHz, up to 60 MHz, or up to 40 MHz. The lower limit of the pulse repetition rate is more practical in a sense of reducing the overall sampling time, but theoretically the data can be taken very slowly if there is some reason to do so.

The device 500 can optionally include an optical microscope 520 for acquiring visual images of cells that are located in the observation zone 506 or elsewhere along the cell analysis pathway 502.

The device 500 can optionally include a cell size measurement tool 522. The cell size measurement tool 522 can be any device capable of measuring the size of cells, including but not limited to, an optical microscope, such as optical microscope 520. In some cases, the optical microscope and the cell size measurement tool 522 are the same subsystem.

In some cases, the autofluorescence spectrometer 510 and the optical microscope 520 can be integrated into a single optical subsystem. In some cases, the autofluorescence spectrometer 510 and the cell size measurement tool 522 can be integrated into a single optical subsystem. While some aspects of the methods described herein can operate by not utilizing the cell size as an input to the convolutional neural network, it may be useful to measure the cell size for other purposes.

The processor 512 is in electronic communication with the spectrometer 510. The processor 512 is also in electronic communication with, when present, the optional cell sorter 508, the optional optical microscope 520, and the optional cell size measurement tool 522.

The non-transitory computer-readable medium 514 has stored thereon instructions that, when executed by the processor, cause the processor to execute at least a portion of the methods described herein. Equations for which the first and second phasor coordinates are inputs can also be stored on the non-transitory computer-readable medium 514. The non-transitory computer-readable medium 514 can be local to the device 500 or can be remote from the device, so long as it is accessible by the processor 512.

The device 500 can be substantially free of fluorescent labels (i.e., the cell analysis pathway 502 does not include a region for mixing the cell(s) with a fluorescent label). The device 500 can be substantially free of immobilizing agents for binding and immobilizing reprogramming intermediate cells.

### EXAMPLE 1

Before this example is described in detail, it should be appreciated that many of the observations described herein are supported by data that can be provided to a patent examiner upon request. In the interest of brevity, much of the raw data and images have been excluded from this description, because these data and images do not provide a better understanding of the invention and merely support some of the statements made and conclusions drawn.

### Materials and Methods

### EPC Isolation and Cell Culture

EPCs were isolated from fresh peripheral human blood that was obtained from healthy donors (Interstate Blood Bank, Memphis, Tenn.). Blood was processed within 24 hours of collection, where hematopoietic progenitor cells were extracted from whole blood using negative selection (RosetteSep; STEMCELL Technologies) and cultured in polystyrene tissue culture plates in erythroid expansion medium (STEMCELL Technologies) for 10 days to enrich for EPCs.

Enriched EPCs from Day 10 were examined by staining with APC Anti-Human CD71 antibody (334107; Biolegend; 1:100) and incubating for 1 hour at room temperature. Data were collected on Attune Nxt flow cytometer and analyzed with FlowJo.

### Micropattern Design and PDMS Stamp Production

First, a template with the feature designs was created in AutoCAD (Autodesk). The template was then sent to the Advance Reproductions Corporation, MA for the fabrication of a photomask, and a 6-inch patterned Si wafer was fabricated by the Microtechnology Core, University of Wisconsin—Madison, Wis. Using soft photolithography techniques, the Si wafer was spin-coated with a SU-8 negative photoresist (MICRO CHEM) and exposed to UV light. The Si mold was then developed for 45 minutes in SU-8 developer (Sigma) which yielded features with a height of 150 μm. The Si mold was then washed with acetone and isopropyl alcohol.

Elastomeric stamps used for microcontact printing were generated by standard soft lithographic techniques. The silicon mold was rendered inert by overnight exposure in vapors of (tridecafluoro-1,1,2,2-tetrahydrooctyl) trichlorosilane. Poly-dimethylsiloxane (Sylgard 184 silicone elastomer base, 3097366-1004, Dow Corning; PDMS) was prepared at a ratio of 1:10 curing agent (Sylgard 184 silicone elastomer curing agent, 3097358-1004, Dow Corning) and degassed in a vacuum for 30 minutes. The PDMS was then poured over the SU-8 silicon mold on a hot plate and baked at 60° C. overnight to create the PDMS stamp.

### μCP Well Plate Construction

Microcontact patterned (μCP) substrates were constructed based on previous studies. In brief, polydimethylsiloxane (PDMS) stamps with 300 μm radius circular features were coated with Matrigel (WiCell Research Institute) for 24 h. After 24 h, the Matrigel-coated PDMS stamp was dried with N₂ and placed onto 35 mm cell culture treated ibiTreat dishes (81156; Ibidi). A 50 g weight was added on top of the PDMS stamps to ensure even pattern transfer from the Matrigel-coated PDMS stamp to the ibiTreat dish. This setup was incubated for 2 h at 37° C. The 35 mm ibiTreat dish was then backfilled with PLL (20 kDa)-g-(3.5)-PEG (2 kDa) (Susos), a graft polymer solution in with a 20 kDa PLL backbone with 2 kDa PEG side chains, and a grafting ratio of 3.5 (mean PLL monomer units per PEG side chain), by using 0.1 mg/mL solution in 10 mM HEPES buffer for 30 min at RT. The ibiTreat dish was then washed with PBS and exposed to UV light for 15 min for sterilization to yield the micropatterned substrate.

### Reprogramming

Day 10 EPCs were electroporated with four episomal reprogramming plasmids encoding Oct4, shRNA knockdown of p53 (#27077; Addgene); Sox2, Klf4 (#27078; Addgene); L-Myc, Lin28 (#27080; Addgene); miR302-367 cluster (#98748; Addgene), using the P3 Primary Cell 4D-Nucleofector Kit (Lonza) and the EO-100 program. Electroporated EPCs were seeded onto micropatterned substrates with erythroid expansion medium (STEMCELL Technologies) at a seeding density of 2000 k cells/dish. Cells were supplemented with ReproTeSR (STEMCELL Technologies) on alternate days starting from Day 3 without removing any medium from the well. On Day 9, the medium was entirely switched to ReproTeSR, and the ReproTeSR medium was changed daily starting from Day 10.

### Isolation of iPSCs

To isolate high-quality iPSC lines, candidate colonies were picked from micropatterns using a 200 μL micropipette tip and transferred to Matrigel-coated polystyrene tissue culture plates in mTeSR1 media (WiCell Research Institute). If additional purification was required, one additional manual picking step with a 200 μL micropipette tip was performed. During picking and subsequent passaging, the culture media was often supplemented with the Rho kinase inhibitor Y-27632 (Sigma-Aldrich) at a 10 μM concentration to encourage cell survival and establish clonal lines. iPSCs obtained from EPCs were maintained in mTeSR1 media on Matrigel-coated polystyrene tissue culture plates and passaged with ReLeSR (STEMCELL Technologies) every 3-5 days. All cells were maintained at 37° C. and 5% CO₂.

### Antibodies and Staining

All cells were fixed for 15 minutes with 4% paraformaldehyde in PBS (Sigma-Aldrich) and permeabilized with 0.5% Triton-X (Sigma-Aldrich) for >4 hours at room temperature before staining. Hoechst (H1399; Thermo Fisher Scientific, Waltham, Mass.) was used at 5 μg/mL with 15 min incubation at room temperature to stain nuclei. Primary antibodies were applied overnight at 4° C. in a blocking buffer of 5% donkey serum (Sigma-Aldrich) at the following concentrations: Anti-Laminin (L9393; Sigma-Alrich) 1:500; TRA-1-60 (MAB4360; EMD Millipore, Burlington, Mass.) 1:100; Nanog (AF1997; R&D Systems) 1:200; CD71 (334107; Biolegend) 1:100. Secondary antibodies were obtained from Thermo Fisher Scientific and applied in a blocking buffer of 5% donkey serum for one hour at room temperature at concentrations of 1:400-1:800. A Nikon Eclipse Ti epifluorescence microscope was used to acquire single 10× images of each micropattern, and a Nikon AR1 confocal microscope was used to acquire 60× stitched images of each micropattern using the z-plane closest to the micropatterned substrate for reprogramming studies.

### Autofluorescence Imaging of NAD(P)H and FAD

Fluorescence lifetime imaging (FLIM) was performed at different time points during reprogramming by an Ultima two-photon microscope (Bruker) composed of an ultrafast tunable excitation laser source (Insight DS+, Spectra-Physics) coupled to a Nikon Ti-E inverted microscope with time-correlated single-photon counting electronics (SPC-150, Becker & Hickl). The laser source enables sequential excitation of NAD(P)H at 750 nm and FAD at 890 nm. NAD(P)H and FAD images were acquired through 440/80 nm and 550/100 nm bandpass filters (Chroma), respectively, using Gallium arsenide phosphide (GaAsP) photomultiplier tubes (PMTs; H7422, Hamamatsu). The laser power at the sample was approximately 3.5 mW for NAD(P)H and 6 mW for FAD. Lifetime imaging using time-correlated single-photon counting electronics (SPC-150, Becker & Hickl) was performed within Prairie View Atlas Mosaic Imaging (Bruker Fluorescence Microscopy) to capture the entire μFeature. Fluorescence lifetime decays with 512-time bins were acquired across 512×512-pixel images with a pixel dwell time of 4.8 μs and an integration period of 60 seconds. Photon count rates were ˜1-5×10⁵ and monitored during image acquisition to ensure that no photobleaching occurred. All samples were placed on a stage-op incubator and illuminated through a 40×/1.15 NA objective (Nikon). The short lifetime of red-blood-cell fluorescence at 890 nm was used as the instrument response function and had a full-width half maximum of 240 ps. A YG fluorescent bead (τ=2.13±0.03 ns, n=6) was imaged daily as a fluorescence lifetime standard.

### Image Analysis

Fluorescence lifetime decays were analyzed to extract fluorescence lifetime components via SPCImage software (Becker & Hickl). A threshold was used to exclude pixels with low fluorescence signals (that is, background). Fluorescence lifetime decays were deconvolved from the instrument response function and fit to a two-component exponential decay model, I(t)=α₁e^{-t/τ}₁+α₂e^{-t/τ}₂+C, where I(t) is the fluorescence intensity as a function of time t after the laser pulse, α₁ and α₂ are the fractional contributions of the short and long lifetime components, respectively (that is, α₁+α₂=1), τ₁ and τ₂ are the short and long lifetime components, respectively, and C accounts for background light. Both NAD(P)H and FAD can exist in quenched (short lifetime) and unquenched (long lifetime) configurations; the fluorescence decays of NAD(P)H and FAD are therefore fit to two components. Fluorescence intensity images were generated by integrating photon counts over the per-pixel fluorescence decays.

Images were analyzed at the single-cell level to evaluate cellular heterogeneity.

A pixel classifier was trained on 15 images using ilastik software (Berg, S. et al. ilastik: interactive machine learning for (bio)image analysis. Nat. Methods 16, 1226-1232 (2019)) to identify the pixels within the nuclei in NAD(P)H images. An object classifier was then used to identify the nuclei in NAD(P)H images using the pixel classifier along with the following parameters: Method=Simple, Threshold=0.3, Smooth=1, Size Filter Min=15 pixels, Size Filter Max=500 pixels. A customized CellProfiler (Carpenter, A. E. et al. CellProfiler: image analysis software for identifying and quantifying cell phenotypes. Genome Biol. 7, R100 (2006)) pipeline was then used to obtain metabolic and nuclear parameters. The CellProfiler pipeline applied the following steps: Primary objects (nuclei) were inputted from ilastik. Secondary objects (cells) were then identified in the NAD(P)H intensity image by outward propagation of the primary objects. Cytoplasm masks were determined by subtracting the nucleus mask from the cell mask. Cytoplasm masks were applied to all images to determine single-cell redox ratio and NAD(P)H and FAD lifetime parameters. A total of 11 metabolic parameters were analyzed for each cell cytoplasm: Optical Redox Ratio, [NAD(P)H], NAD(P)Hα₁, NAD(P)Hτ₁, NAD(P)Hτ₂, NAD(P)Hτₘ, [FAD], FADα₁, FADτ₁, FADτ₂, FADτₘ. A total of 8 nuclear parameters were analyzed for each nucleus: Area, Perimeter, MeanRad, NSI, Solidity, Extent, #Neigh, 1stNeigh.

Representative images of the optical redox ratio (fluorescence intensity of NAD(P)H divided by the summed intensity of NAD(P)H and FAD) and mean fluorescence lifetimes (τₘ=α₁τ₁+α₂τ₂) of NAD(P)H and FAD were computed using the Fiji software.

### UMAP Clustering

Clustering of cells across EPCs, IMs, and iPSCs was represented using Uniform Manifold Approximation and Projection (UMAP). UMAP dimensionality reduction (McInnes, L., Healy, J., Saul, N. & Groβberger, L. UMAP: Uniform Manifold Approximation and Projection. J. Open Source Softw. 3, 861 (2018)) was implemented using R on all 11 OMI parameters (optical redox ratio, NAD(P)Hτₘ, τ₁, τ₂, α₁, α₂; FADτₘ, τ₁, τ₂, α₁, α₂) and/or all 8 nuclear parameters (Area, Perimeter, MeanRad, NSI, Solidity, Extent, #Neigh, 1stNeigh) for projection in 2D space. The following parameters were used for UMAP visualizations: “n_neighbors”: 20; “min_dist”: 0.3, “metric”: Jaccard, “n_components”: 2.

### Z-Score Hierarchical Clustering

Z-score of each metabolic and nuclear parameter for each cell was calculated. Z-score=(μ_{observed}−μ_{row})/σ_{row}, where μ_{observed} is the mean value of each parameter for each cell; μ_{row} is the mean value of each parameter for all cells together, and σ_{row} is the standard deviation of each parameter across all cells. Heatmaps of z-scores for all OMI variables were generated to visualize differences in each parameter between different cells. Dendrograms show clustering based on the similarity of average Euclidean distances across all variable z-scores. Heatmaps and associated dendrograms were generated in Python.

### Classification Methods

Random forest, Simple Logistic, k-nearest neighbor (IBk), and naïve bayes classification methods were trained to classify reprogramming cells into EPCs, IMs, and iPSCs using Weka software (Holmes, G., Donkin, A. & Witten, I. H. WEKA: a machine learning workbench. in Proceedings of ANZIIS '94—Australian New Zealnd Intelligent Information Systems Conference 357-361 (IEEE, 1994). doi:10.1109/ANZIIS.1994.396988). All data were randomly partitioned into training and test datasets using 15-fold cross-validation for training and test proportions of 93.3% (1994 cells) and 6.7% (143 cells), respectively. Each model was replicated 100 times; new training and test data were generated before each iteration. Parameter weights for metabolic and nuclear parameters were extracted using the GainRatioAttributeEval function in Weka to determine the contribution of each variable to the trained classification models. One-vs-Rest receiver operating characteristic (ROC) curves were generated to evaluate the classification model performance on the classification of test set data and are the average of 100 iterations of data that was randomly selected from training and test sets. All of the ROC curves displayed were constructed from the test datasets using the model generated from the training data sets.

### Karyotyping

Cells cultured for at least 5 passages were grown to 60-80% confluence and shipped for karyotype analysis to WiCell Research Institute, Madison, Wis. G-banded karyotyping was performed using standard cytogenetic protocols. Metaphase preparations were digitally captured with Applied Spectral Imaging software and hardware. For each cell line, 20 GTL-banded metaphases were counted, of which a minimum of 5 was analyzed and karyotyped. Results were reported in accordance with guidelines established by the International System for Cytogenetic Nomenclature 2016.

### Statistics

p-values were calculated using the non-parametric Kruskal-Wallis test for multiple unmatched comparisons with GraphPad Prism software. Statistical tests were deemed significant at α≤0.05. Technical replicates are defined as distinct μFeatures within an experiment. Biological replicates are experiments performed with different donors. No a priori power calculations were performed.

### Results

Establishment of reprogramming on microcontact printed substrates.

We first designed a microcontact printed (μCP) substrate to spatially control the adhesion of EPCs undergoing reprogramming. The μCP substrate is formed by Matrigel coating of 300 μm radius circular regions, referred to as μFeatures, on a 35 mm ibiTreat dish that allows for cell adhesion. The remaining regions of the dish are then backfilled with polycationic graft copolymer, PLL-g-PEG, that resists protein adsorption and prevents cell adhesion in these regions. The ibiTreat dishes are made of gas-permeable material, enabling maintenance of carbon dioxide or oxygen exchange during cell culture and have high optical quality. These properties make the dishes suitable for two-photon microscopy during reprogramming. To verify the stability of the Matrigel-coated circular regions, we immunostained for laminin, a major component of Matrigel. Fluorescence imaging showed laminin consistently within the circular μFeatures indicating uniform patterning of Matrigel. We next assessed the ability of the μCP substrates to enable cell attachment by seeding two different cell types i.e., human dermal fibroblasts (HDFs) and H9 human embryonic stem cells (H9 ESCs). We observed that both HDFs and ESCs remained viable, attached, and confined to the circular μFeatures indicating that the μCP substrates enable spatial control of cell attachment.

Next, we isolated peripheral blood mononuclear cells (PBMCs) from peripheral blood of healthy human donors and further enriched them for EPCs. We examined the enrichment of EPCs by flow cytometry with erythroid cell surface marker CD71. Flow cytometry confirmed the presence of enriched EPCs with flow cytometry showing that >98% of the cells expressed CD71 on day 10 of culture.

To initiate reprogramming, we electroporated the EPCs with four episomal reprogramming plasmids, encoding Oct4, shRNA knockdown of p53, Sox2, Klf4, L-Myc, Lin28, and miR302-367 cluster; and seeded them onto μCP substrates. We assessed the ability of the μCP substrates to sustain long-term reprogramming studies by performing high-content imaging to track individual μFeatures (>30 μFeatures per 35 mm dish) longitudinally at multiple timepoints over the ˜3-week reprogramming time course. Day 22 was picked as the reprogramming endpoint because there were several iPSC colonies at this timepoint without significant outgrowth within a μFeature, enabling analysis at single-cell resolution. While starting EPCs are non-adherent, reprogramming intermediate cells (IMs) and endpoint iPSCs adhere to the circular μFeatures within the μCP substrates indicating that μCP substrates can support reprogramming of EPCs. Overall, the μCP platform provides unique spatial control over reprogramming cells and enables high-content quantitative imaging of reprogramming.

OMI reveals distinct metabolic changes during reprogramming.

Metabolic state plays an important role in regulating reprogramming and pluripotency of iPSCs and can be non-invasively monitored via OMI. NADH is an electron donor and FAD is an electron acceptor, both present in all cells as coenzymes and provide energy for metabolic reactions. For example, glycolysis in the cytoplasm generates NADH and pyruvate, while OXPHOS consumes NADH and produces FAD. Autofluorescence imaging of NADH and FAD is thus dynamically responsive to the oxidation-reduction state of a cell and is influenced by many reactions.

We tracked the autofluorescence dynamics of NAD(P)H and FAD by performing OMI on μCP substrates at different time points during EPC reprogramming. In these images, the nucleus remains dark as NAD(P)H is primarily located in cytosol and mitochondria, and FAD is primarily located in mitochondria. The NAD(P)H images were used as inputs for ilastik software (citation above) to identify the nuclei. The identified nuclei were then used as an input for high-content CellProfiler software (citation above) pipeline to segment the cytoplasm, and measure various metabolic and nuclear parameters. Overall, a total of 11 metabolic parameters (Optical Redox Ratio, [NAD(P)H], NAD(P)Hα₁, NAD(P)Hτ₁, NAD(P)Hτ₂, NAD(P)Hτₘ, [FAD], FADα₁, FADτ₁, FADτ₂, FADτₘ, and 8 nuclear parameters (Molugu, K. et al. Tracking and Predicting Human Somatic Cell Reprogramming Using Nuclear Characteristics. Biophys. J. 118, 2086-2102 (2020)) (Area, Perimeter, MeanRad, NSI, Solidity, Extent, #Neigh, 1stNeigh) were measured by the analysis pipeline. Additionally, immunofluorescence labeling verified the cell type at these different time points, i.e., EPCs (CD71⁺, Nanog⁻), IMs (CD71⁻, Nanog⁻), and iPSCs (CD71⁻, Nanog⁺). NAD(P)H and FAD autofluorescence imaging revealed metabolic differences between starting EPCs, intermediates (IM), and iPSCs.

We observed a significant increase in the optical redox ratio (iPSC>IM>EPC) during the process of reprogramming, indicating that EPCs are more oxidized than IMs and iPSCs. Additionally, we noted that patterned IMs and iPSCs have significantly higher optical redox ratios as compared to their non-patterned counterparts. This observation is consistent with previous studies which show that mechanical cues can regulate their relative use of glycolysis and may require further investigation.

Next, we observed that NAD(P)H and FAD lifetime components undergo biphasic changes during the progress of reprogramming. FAD lifetime components undergo a more significant change relative to the NAD(P)H components. The fraction of protein-bound FAD (FADτ₁) undergoes a decrease from EPCs to IMs and then an increase from IMs to iPSCs, which could be reflective of the OXPHOS burst. FADτₘ is inversely related to FADα₁ and therefore undergoes a biphasic change that is opposite to that of FADτₘ. Similar biphasic changes occur in nuclear parameters during reprogramming which is consistent with our previous study.

We noted that H9 ESCs have metabolic and nuclear parameters similar to iPSCs, as expected. Fibroblasts had metabolic parameters significantly different from EPCs (FIG. 1 Supplement 2). This could be because 1) fibroblasts are adherent while starting EPCs are non-adherent, 2) fibroblasts and EPCs have different proliferation rates and energy needs.

Taken together, autofluorescence imaging of NAD(P)H and FAD showed significant changes during reprogramming.

OMI enables the classification of reprogramming cells with high accuracy.

Uniform Manifold Approximation and Projection (UMAP) (citation above), a dimension reduction technique, was used to visualize how cells cluster from metabolic and nuclear parameters onto 2D space. Neighbors were defined through the Jaccard similarity coefficient computed across the metabolic parameters and nuclear parameters. UMAP was chosen over t-distributed stochastic neighbor embedding (t-SNE) since UMAP separated EPCs, IMs, and iPSCs better than t-SNE. Moreover, UMAP has a higher speed and ability to include non-metric distance functions and preserves the global structure of the data.

We used UMAP to visualize how cells cluster exclusively from the 11 metabolic parameters and exclusively from the 8 nuclear parameters. While these UMAP representations revealed separation of EPCs, IMs, and iPSCs; UMAP generated using both metabolic and nuclear parameters provided cleaner separation between EPCs, IMs, and iPSCs. We also plotted a heatmap representation of the z-score of metabolic and nuclear parameters at the donor level to examine donor heterogeneity. In summary, clustering of 11 metabolic and 8 nuclear parameters by using UMAP and z-score heatmap clustering showed that EPCs, IMs, and iPSCs can be distinguished based on these parameters.

Next, classification models were developed based on 11 metabolic and 8 nuclear parameters to predict the reprogramming status of cells, i.e., EPCs, IMs, or iPSCs. Supervised machine learning classification (Naïve Bayes, K-nearest neighbor) and regression algorithms (logistic regression, and random forest—see, Amancio, D. R. et al. A Systematic Comparison of Supervised Classifiers. PLoS ONE 9, e94137 (2014)) were implemented to test the prediction accuracy for iPSCs when all the metabolic and nuclear parameters are used. To protect against over-fitting, the classification models were trained using 15-fold cross-validation on single-cell data from three different donors with reprogramming status assigned from morphological characteristics and tested on data with the same cell CD71 and Nanog staining validation from three donors (completely independent and non-overlapping observations). One-vs-Rest receiver operator characteristic (ROC) curves of the test data revealed highest classification accuracy for predicting iPSCs (area under the curve AUC=0.993), IMs (AUC=0.993) and EPCs (AUC=0.999) when random forest classification model is used. We thus used the random forest classification model for further analysis in this study.

Gain ratio analysis revealed that FAD lifetime components, FADα₁, FADτ₁, and FADτₘ, are the most important parameters for classifying the reprogramming status of cells. This is consistent with the observation that FAD lifetime components are significantly different among EPCs, IMs, and iPSCs. We then plotted the accuracy score as a function of the number of parameters (chosen based on the gain ratio values for random forest classifier) used for classification. This plot revealed that the accuracy score increases with the number of parameters until 8 parameters and plateaus thereafter. We additionally noted that using only FAD lifetime variables (FADτₘ, τ₁, τ₂, α₁; collected in the FAD channel alone), high classification accuracy can be achieved for predicting iPSCs (area under the curve AUC=0.944), IMs (AUC=0.968) and EPCs (AUC=0.998). Using only FAD lifetime parameters ensures minimal imaging time of 2.5 min per μFeature, and no additional reliance on intensity parameters which are associated with higher variability due to the confounding factors of intensity levels (throughput due to laser power, detector gain, and inner filter effects). Hence, FAD lifetime parameters alone are sufficient to predict the reprogramming status of cells.

Pseudotemporal ordering of single cells reveals heterogeneous cell populations.

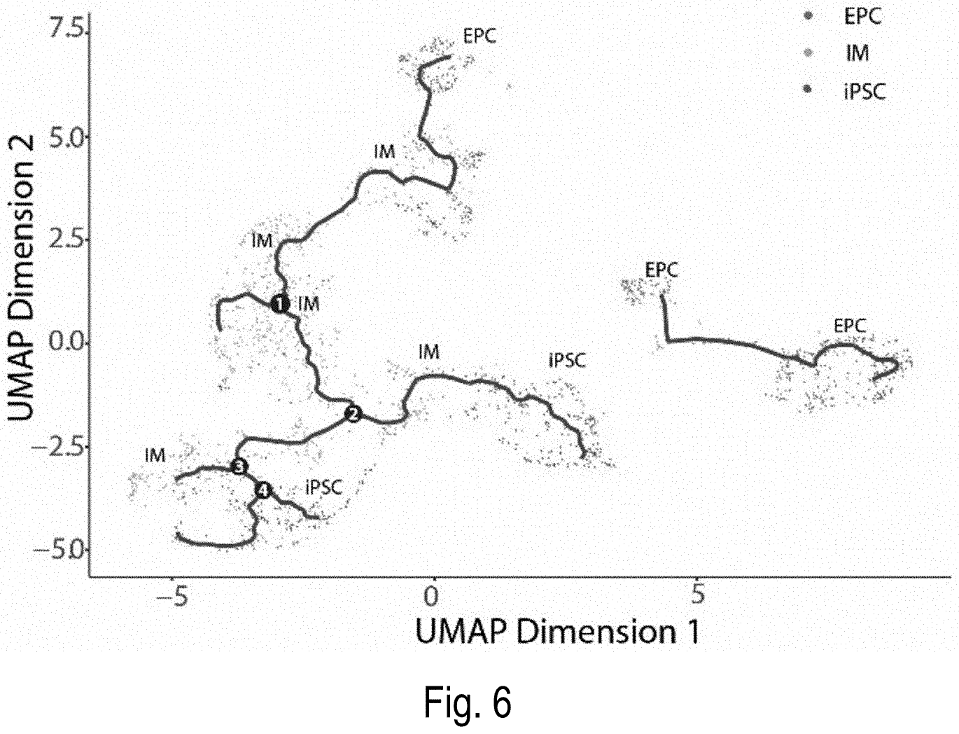

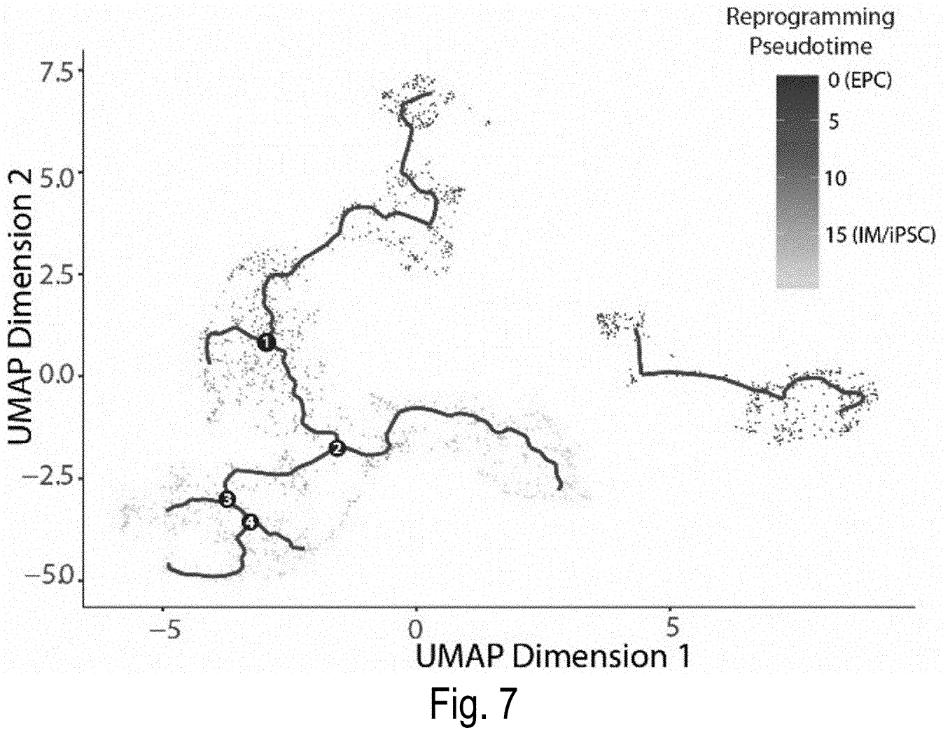

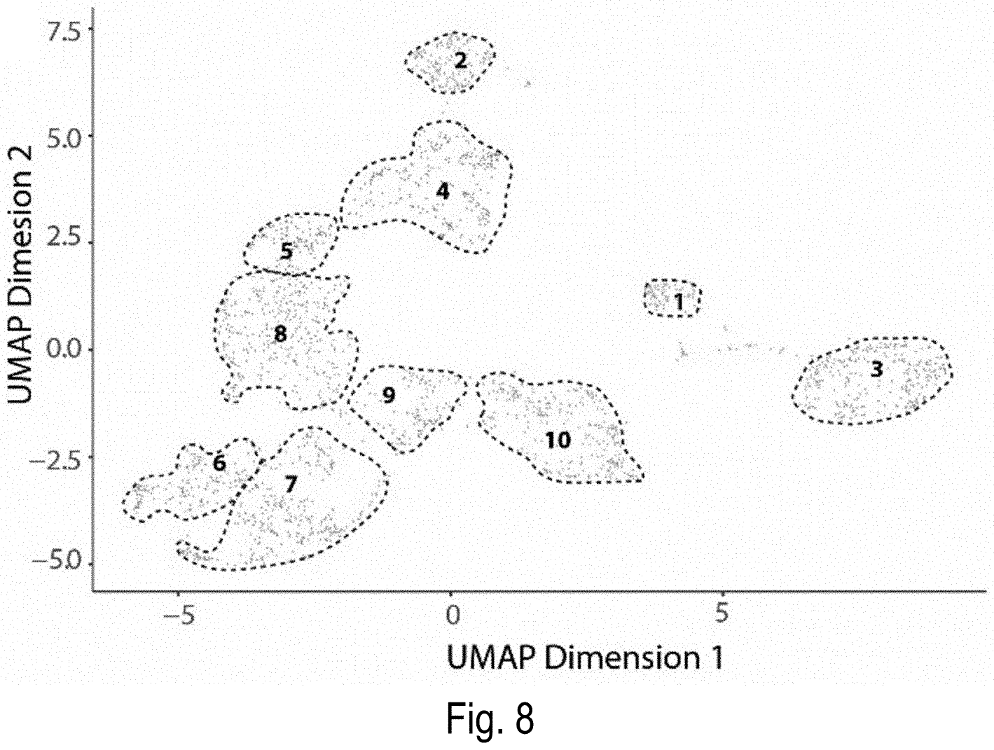

To study the heterogeneity of reprogramming μFeatures, we used 11 metabolic and 8 nuclear parameters to construct pseudotime single-cell trajectories of cellular reprogramming using the Monocle3 program (see, Trapnell, C. et al. The dynamics and regulators of cell fate decisions are revealed by pseudotemporal ordering of single cells. Nat. Biotechnol. advance online publication, (2014) and Qiu, X. et al. Reversed graph embedding resolves complex single-cell trajectories. Nat. Methods 14, 979-982 (2017)), which is a trajectory inference method that learns combinatorial changes which each cell must go through as a part of the reprogramming process and subsequently places each cell at its proper location in the trajectory. The trajectory built using this method consisted of EPCs, IMs, and iPSCs distributed across 10 clusters, 4 branching events, and a disconnected branch (FIG. 6-8). Notably, the pseudotime trajectory colored by actual reprogramming time points showed that pseudotime progresses in line with the actual reprogramming timeline (FIG. 7).

The starting EPCs were heterogeneous and occupied three clusters (clusters: 1, 2, 3). While cluster 2 consists of starting EPCs that undergo reprogramming, clusters 1 and 3 constituted the disconnected branch with EPCs may not easily be permissive to reprogramming. iPSCs predominantly occupied two clusters (clusters: 7, 10) irrespective of the reprogramming timepoint, while IMs belonged to several clusters (clusters: 4, 5, 6, 8, 9) with clusters 6 and 8 concentrated at the unsuccessful reprogramming branches (FIG. 3C). Overall, this single-cell trajectory map is indicative of reprogramming heterogeneity i.e., cells that advance right at branch points 1, 2, and 3 (FIG. 7, 8) completely reprogram to iPSCs within 25 days of reprogramming initiation while cells that proceed left at branch points 1 and 3 (FIG. 7, 8) remain at the intermediate stage.

Subsequent heatmap analysis on the clusters in the single-cell reprogramming trajectory map revealed that the clusters exhibited correlation patterns based on their reprogramming status i.e., EPCs (clusters: 2) have a high correlation to early IMs (cluster: 4), while late IMs (clusters: 5,6,8,9) demonstrate high correlation to iPSCs (clusters: 7). When we compared IMs that undergo reprogramming (cluster: 9) and the IMs that do not reprogram to iPSCs (cluster: 6), we noted differences in their NAD(P)H lifetime components, indicating that these parameters might play a role in determining reprogramming cell fate. To further examine the parameters that distinguished the cell clusters, we performed spatial correlation analysis using Moran's I, which is a statistic that tells whether cells at nearby positions on a trajectory will have similar (or dissimilar) expression levels for the parameter being tested. When the parameters were ranked by Moran's I, FAD lifetime parameters (FADτ₁, τ₂, τₘ) were found to be the most important in distinguishing clusters followed by NAD(P)H lifetime parameters (NAD(P)Hτ₂, α₁, τ₁). This result is consistent with high gain ratio values for FAD lifetime parameters and the observation FAD lifetime parameters are significantly different among EPCs, IMs, and iPSCs.

Overall, while FAD parameters are important in distinguishing EPCs, IMSs, and iPSCs; NAD(P)H parameters are key for determining the eventual reprogramming fate of cells. When we plotted the identified important metabolic parameters as a function of pseudotime, we observed that they undergo biphasic changes during reprogramming which could be representative of the OXPHOS burst. These pseudotime trajectory plots provided increased temporal resolution as compared to our previous plots. Taken together, our reprogramming trajectory analyses provided insights into reprogramming heterogeneity at a high temporal resolution and single-cell resolution.

### Isolation of High-Quality iPSCs

The terminal goal of any reprogramming platform is to successfully isolate iPSCs that can be used for downstream applications. Using the combination of OMI, μCP platform, and machine learning models developed in this study, we were able to successfully isolate high-quality iPSCs.

First, we tracked the metabolic and nuclear parameters of μFeatures throughout the reprogramming time course using OMI. Second, we employed our random forest classification model to predict the reprogramming status of the tracked μFeatures. Third, we inferred the pseudotimes during the reprogramming time course to monitor the progress of the μFeatures along the reprogramming trajectory. Finally, we performed immunostaining on the μFeatures, which showed that the reprogramming status predictions made by the machine learning models correlated well with the actual staining.

We then isolated iPSCs from the μCP platform based on the predictions made by the random forest classification model. The physical separation of micropatterns from one another, combined with a high fraction of predicted iPSC cells, even up to 100%, throughout the μFeature resulted in easy picking and isolation of completely reprogrammed iPSCs. We further confirmed that the isolated iPSCs expressed pluripotency markers and showed no genomic abnormalities, indicating that our reprogramming platform can be used to generate genetically-stable iPSC lines.

### Discussion

Here, we report a non-invasive, high-throughput, quantitative, and label-free imaging platform to predict the reprogramming outcome of EPCs by combining micropatterning, live-cell autofluorescence imaging, and automated machine learning. We are able to predict the reprogramming status of EPCs at any timepoint during reprogramming with a prediction accuracy of ˜95% and model performance of ˜0.99 (AUC of ROC) using a random forest classification model with 11 metabolic parameters and 8 nuclear parameters. Additionally, we provide a single cell roadmap of EPC reprogramming, which reveals diverse cell fate trajectories of individual reprogramming cells (FIGS. 6-8).

Recent evidence indicates that metabolic changes during reprogramming include decreasing OXPHOS and increasing glycolysis, along with a transient hyper-energetic metabolic state, called OXPHOS burst. This OXPHOS burst occurs at an early stage of reprogramming and shows characteristics of both high OXPHOS and high glycolysis, which could be a regulatory cue for the overall shift of reprogramming. These changes are accompanied by alterations in the amounts of corresponding metabolites and have been confirmed by genome-wide analyses of gene expression, protein levels, and metabolomic profiling. The shifts in cellular metabolism affect enzymes that control epigenetic configuration, which can impact chromatin reorganization and provide a basis for changes in nuclear morphology as well as gene expression during reprogramming. Consistent with these studies, the redox ratio increases during reprogramming, which could be indicative of increased glycolysis during reprogramming.

The changes in NAD(P)H and FAD lifetime parameters that occur during reprogramming could reflect changes in quencher concentrations, such as oxygen, tyrosine, or tryptophan, or changes in local temperature and pH. Specifically, the biphasic changes in the metabolic and nuclear parameters could be transient increased due to the increased production of ROS by mitochondria during OXPHOS burst. The generated ROS further serves as a signal to activate Nuclear Factor (erythroid derived 2)-like-2 (NRF-2), which then induces hypoxia-inducible factors (HIFs) that promote glycolysis during reprogramming by increasing the expression levels of the glycolysis-related genes.

Moreover, the importance of FAD parameters for distinguishing various reprogramming cell types could point to the significant changes in the mitochondrial environment during reprogramming. The differences in NAD(P)H lifetime parameters between IMS that successfully undergo reprogramming and the ones that do not, may suggest the role of NAD(P)H in impacting reprogramming barriers and warrants further investigation.

The classification analysis revealed that models trained on all 11 metabolic and 8 nuclear parameters yielded the highest accuracy for the classification of reprogramming status of cells. Random forest classification using only FAD lifetime parameters yielded comparatively high ROC AUC values. Additionally, FAD lifetime parameters were more accurate for predicting reprogramming status than using nuclear parameters alone, which can be obtained using widefield or confocal fluorescence microscopy. Imaging only FAD lifetime parameters instead of imaging all the parameters significantly reduced the time of imaging from 7 min to 2.5 min per μFeature. This is especially helpful when multiple μFeatures are trying to be assessed for iPSC quality at a manufacturing scale and eliminates the variability associated with intensity measurements.

Our single-cell reprogramming trajectory maps built based on metabolic and nuclear parameters (FIGS. 6 to 8) could indicate that the reprogramming process is proceeding by a combination of elite and stochastic models. While there seems to be a fraction of starting EPCs that are refractory towards reprogramming supporting the elite model of reprogramming, there is also a fraction of intermediate cells at various stages of reprogramming that do not completely reprogram to iPSCs corroborating the stochastic model of reprogramming.

Much of the current work to understand the heterogeneity during reprogramming relies on bulk analysis or single-cell analysis techniques. While bulk samples obscure variability in both the starting cell population and during fate conversion, owing to the variable kinetics and low efficiency of reprogramming; single-cell techniques disrupt the cells' microenvironment, resulting in significant changes in the biophysical properties of cells undergoing reprogramming. Our reprogramming platform overcomes these challenges by using the combination of μCP platform, OMI, and monocle algorithm. Firstly, the μCP platform ensures an intact microenvironment for reprogramming cells while also allowing for analysis at the single-cell level. Secondly, OMI has several spatial and temporal resolution advantages compared with traditional assays enabling greater insights into reprogramming heterogeneity. OMI can be performed at high resolution to enable measurements at single-cell level, is non-destructive allowing for spatial integrity measurements of neighboring cells, and also has a high temporal resolution enabling the time-course study of reprogramming. Finally, pseudotime trajectories using monocle algorithm overcomes the problems of reprogramming trajectories built based on absolute time points which disregard the asynchrony of the reprogramming process. Overall, these methods could aid in the identification of somatic cells or early reprogramming cells which are refractory towards reprogramming and thus be used to increase the success rate of iPSC generation from patient-derived primary cells or cell lines.

Our reprogramming platform could contribute to the long-term commercial success of iPSC-derived therapies by migrating the iPSC manufacturing process from laborious, time-consuming, error-prone high-risk lab bench protocol to an industrial-scale, GMP-compliant manufacturing system. Firstly, the μCP platform involves direct ECM printing onto optically clear substrates and does not involve any gold coating, unlike traditional microcontact printing methods, making the process cost-effective and simpler by eliminating the need for cleanroom access. We were also able to isolate fully pluripotent iPSCs without any genomic abnormalities using this μCP platform, indicating that this platform could be adapted for biomanufacturing of GMP-grade iPSCs. Secondly, we used erythroid progenitor cells isolated from peripheral blood as the starting cell type for reprogramming due to their lack of genomic rearrangements and demonstrated reprogramming ability. Moreover, blood collection is a minimally invasive procedure, and collected cells are naturally replaced as the tissue is self-renewing, making it suitable for generating iPSCs. Thirdly, we used a combination of oriP/EBNA-based viral-free non-integrating episomal reprogramming plasmids described previously, that avoid the safety concerns surrounding the use of integrating viral vectors. Moreover, these episomes are lost at around 5% per cell generation due to defects in plasmid synthesis and partitioning and thus, iPSCs devoid of plasmids can easily be isolated for clinical applications. We also used xeno-free components for feeder-free reprogramming and maintenance of iPSCs to eliminate the inconsistencies arising from the undefined nature of xeno-components and ensures μCP compliance. Finally, the autofluorescence imaging technique is label-free, unlike other methods to study metabolism like electron microscopy, immunocytochemistry, and colorimetric metabolic assays. It also enables non-destructive real-time monitoring of live cells with lower sample phototoxicity compared to single-photon excitation. Taken together, the processes of μCP platform fabrication, reprogramming, autofluorescence imaging, iPSC identification based on machine learning models and iPSC isolation can all be automated, and be extended to different reprogramming methods like mRNA, Sendai virus; to other starting cell types like fibroblasts, keratinocytes; to other parameters (cell morphology and mitochondrial structure and to other processes like differentiation; making it an attractive platform for biomanufacturing of industrial-scale iPSCs and iPSC-derived cells.

Overall, we developed a high-throughput, non-invasive, rapid, and quantitative method to predict the reprogramming status of cells and study reprogramming heterogeneity. Our studies indicate that OMI can predict the reprogramming status of cells, which could enable real-time monitoring during iPSC manufacturing, thereby aiding in the identification of high-quality iPSCs in a timely and cost-effective manner. Similar technologies could impact other areas of cell manufacturing such as direct reprogramming, differentiation, and cell line development, and thus contribute towards the rapid advancement of regenerative medicine and precision medicine applications from bench to bedside.

We claim:

1. A somatic cell reprogramming tracking device comprising:
a cell analysis observation zone adapted to receive a reprogramming intermediate cell and to present the reprogramming intermediate cell for individual autofluorescence interrogation;
an autofluorescence spectrometer configured to acquire an autofluorescence data set for the reprogramming intermediate cell located in the cell analysis observation zone, the autofluorescence spectrometer comprising a light source, a photon-counting detector, and photon-counting electronics;
a processor in electronic communication with the autofluorescence spectrometer; and
a non-transitory computer-readable medium accessible to the processor and having stored thereon instructions that, when executed by the processor, cause the processor to:
a) receive the autofluorescence data set; and
b) identify a current reprogramming status of the reprogramming intermediate cell based on a current reprogramming prediction, wherein the current reprogramming prediction is computed using at least a portion of the autofluorescence data set, wherein the current reprogramming prediction is computed using at least one metabolic endpoint of the autofluorescence data set and optionally at least one nuclear parameter as an input, wherein the at least one metabolic endpoint includes flavin adenine dinucleotide (FAD) mean fluorescence lifetime (τₘ), the FAD shortest lifetime amplitude component (α₁), FAD shortest fluorescence lifetime component (τ₁), FAD longest fluorescence lifetime component (τ₂), or a combination thereof.

2. The somatic cell reprogramming tracking device of claim 1, wherein the instructions, when executed by the processor, cause the processor to: c) identify a future reprogramming status of the reprogramming intermediate cell based on a pseudotime trajectory of cellular reprogramming that is based off machine learning analysis of acquired autofluorescence data sets for reprogramming intermediate cells having a known reprogramming status over the course of the pseudotime trajectory.

3. The somatic cell reprogramming tracking device of claim 2, wherein the instructions, when executed by the processor, cause the processor to: c) identify the future reprogramming status of the reprogramming intermediate cell based on the current reprogramming prediction and the pseudotime trajectory of cellular reprogramming.

4. A method of characterizing somatic cell reprogramming progression, the method comprising:
a) optionally receiving a population of reprogramming intermediate cells having unknown reprogramming status;
b) acquiring an autofluorescence data set from a reprogramming intermediate cell of the population of reprogramming intermediate cells; and
c) identifying a current reprogramming status of the reprogramming intermediate cell based on a current reprogramming prediction, wherein the current reprogramming prediction is computed using at least a portion of the autofluorescence data set, wherein the current reprogramming prediction is computed using at least one metabolic endpoint of the autofluorescence data set and optionally at least one nuclear parameter as an input, wherein the at least one metabolic endpoint includes flavin adenine dinucleotide (FAD) mean fluorescence lifetime (τₘ), the FAD shortest lifetime amplitude component (α₁), FAD shortest fluorescence lifetime component (τ₁), FAD longest fluorescence lifetime component (τ₂), or a combination thereof.

5. The method of claim 4, the method further comprising:
d) identifying a future reprogramming status of the reprogramming intermediate cell based on a pseudotime reprogramming pathway map that is based off machine learning analysis of acquired autofluorescence data sets for reprogramming intermediate cells having a known reprogramming status over the course of a pseudotime trajectory.

6. The method of claim 5, wherein the identifying the future reprogramming status is further based on the current reprogramming prediction.

7. A method of making a pseudotime reprogramming pathway map, the method comprising:
a) receiving autofluorescence data sets and optionally nuclear data sets for a plurality of reprogramming intermediate cells, the autofluorescence data sets corresponding to pseudotime points along a pseudotime line of reprogramming;
b) constructing pseudotime single-cell trajectories for each of the plurality of reprogramming intermediate cells based on the received autofluorescence data sets associated with each of the plurality of reprogramming intermediate cells, the pseudotime single-cell trajectories each including a current reprogramming prediction associated with each of the predetermined pseudotime points, the current reprogramming prediction is computed using at least a portion of the autofluorescence data sets and optionally using at least a portion of the nuclear data sets, wherein the current reprogramming prediction is computed using at least one metabolic endpoint of at least one of the autofluorescence data sets and optionally at least one nuclear parameter of at least one of the nuclear data sets as an input, wherein the at least one metabolic endpoint includes flavin adenine dinucleotide (FAD) mean fluorescence lifetime (τₘ), the FAD shortest lifetime amplitude component (α₁), FAD shortest fluorescence lifetime component (τ₁), nicotinamide adenine dinucleotide and/or reduced nicotinamide dinucleotide phosphate adenine dinucleotide (NAD(P)H) shortest lifetime amplitude component (α₁), NAD(P)H shortest fluorescence lifetime component (τ₁), NAD(P)H longest fluorescence lifetime component (τ₂), or a combination thereof;
c) compiling the constructed pseudotime single-cell trajectories into a single compiled data set;
d) identifying clusters, branching events, and/or disconnected branches within the single compiled data set; and
e) identifying correlation within the single compiled data set between the clusters, branching events, and/or disconnected branches and current or future reprogramming status, thereby producing the pseudotime reprogramming pathway map for use in predicting future reprogramming based on the correlation.

8. The system of claim 1, wherein the at least one metabolic endpoint includes a redox ratio.

9. The system of claim 1, wherein the at least one metabolic endpoint includes NAD(P)Hτ₁.

10. The system of claim 1, wherein the at least one metabolic endpoint includes FADτ₁.

11. The system of claim 1, wherein the at least one metabolic endpoint includes FADτ₂.

12. The system of claim 1, wherein the at least one metabolic endpoint includes FADα₁.

13. The system of claim 1, wherein the at least one metabolic endpoint includes FADτₘ.

14. The system of claim 1, wherein the current reprogramming prediction is computed using the at least one metabolic endpoint and the at least one nuclear parameter as the input.

15. The system of claim 14, wherein the at least one nuclear parameter includes an area of the nucleus of the reprogramming intermediate cell.

16. The system of claim 14, wherein the at least one nuclear parameter includes a perimeter of the nucleus of the reprogramming intermediate cell.

17. The system of claim 14, wherein the at least one nuclear parameter includes a mean distance of any pixel within the nucleus of the reprogramming intermediate cell to a closest pixel outside of the nucleus.

18. The system of claim 14, wherein the at least one nuclear parameter includes a proportion of pixels located in a convex hull that are also located within the nucleus of the reprogramming intermediate cell.

19. The system of claim 14, wherein the at least one nuclear parameter includes a proportion of image pixels that are located in a bounding box that are also located within the nucleus of the reprogramming intermediate cell.

20. The system of claim 14, wherein the at least one nuclear parameter includes a distance from the reprogramming intermediate cell nucleus to the closest object and/or the closest other nucleus.
